(19) United States
(12) Patent Application Publication
(10) Pub. No.: US 2009/0252290 A1
(43) Pub. Date: Oct. 8, 2009

(54) IN BORE CT LOCALIZATION MARKING LASERS

(75) Inventors: Leonard Plut, Mentor, OH (US); Marc A. Chappo, Elyria, OH (US); Mark R. Pepelea, Aurora, OH (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., Eindhoven (NL)

(21) Appl. No.: 11/720,882

(22) PCT Filed: Dec. 5, 2005
(86) PCT No.: PCT/IB05/54059

§ 371 Date: Jun. 5, 2007

Related U.S. Application Data
(60) Provisional application No. 60/634,581, filed on Dec. 9, 2004.

(51) Int. Cl. A61N 5/10; A61B 6/08

(52) U.S. Cl. 378/65; 378/206

(57) ABSTRACT

A diagnostic imaging system includes a stationary gantry (20) which defines a subject-receiving bore (26). First and second lasers (66, 68) are firmly mounted to the stationary gantry (20). A saggital laser (48) is mounted overhead to project a longitudinal line (58) on a top of the subject in a vertical plane (60) which is parallel to an axial direction (Z). A couch (36) moves a subject into the bore (26)to generate an image of a region of interest and out of the bore for marking. A user segments the image to outline at least an organ. An isocenter (94) of the segmented organ is determined. At least one of the saggital, first and second lasers (48, 66, 68) are adjusted concurrently with adjusting the couch (36) such that laser lines (58, 76, 78) projected by the saggital, first and second lasers (48, 66, 68) intersect the determined isocenter (94). The saggital, first and second lasers (48, 66, 68) laser mark the subject.

The present invention relates to the diagnostic imaging arts. It finds particular application in conjunction with the oncological studies and will be described with particular reference thereto. However, it is to be appreciated that the present invention is applicable to a wide range of diagnostic imaging modalities and to the study of a variety of organs for a variety of reasons.

In oncological planning, the oncologist typically generates a CT image or a plurality of x-ray, projection images of a region to be treated. One of the priorities in oncological procedures is to accurately, and with a reliable repeatability, align an energetic x-ray photon beam with the internal tumor. If the selected trajectory is not accurately located, the x-ray beam will treat most of the tumor, but leave a segment un-irradiated while damaging healthy tissue. Conversely, some tissue is easily damaged by radiation and dense tissue, e.g. bone absorbs a significant portion of the radiation altering the dose. The trajectories are selected to miss these tissues, but often need to come close to them to reach the target with specified margins. If the trajectory is slightly off, these tissues could be damaged or the dose unknowingly altered.

It is critical to position a patient with respect to the radiation apparatus such that the center of the zone to be irradiated coincides with the isocenter of the radiation apparatus. The CT simulators from Philips Medical Systems typically use absolute patient marking. In absolute marking, a CT scan is performed and the center of the treatment region is determined while the patient remains on the couch. The couch is moved to position the tumor outside of the bore at a point of intersection of three lasers which are also positioned outside of the bore. A sagittal laser line is projected from the top and crosshair laser lines are projected from either side of the patient couch. The position of the crosshairs and the intersection of the side and top lasers on the patient are marked to identify the location of the tumor.

Because a physician needs to have an access to the patient, the three lasers are installed a set distance from the front of the gantry. In this approach, the side lasers, transverse and coronal, are co-planer and are typically mounted to the floor in stanchions or on the wall. A sagittal assembly is mounted to the ceiling or on the wall opposite the foot end of the patient support.

However, the mounting of the marking lasers in front of the gantry is often difficult in terms of exact placement in relation to the gantry due to obstructions within the room. Additionally, the side lasers are mounted at a fixed distance of 500-700 mm from the scan plane. The marking accuracy due to variations in the patient support (differential sag between the marking plane and the scan plane) is changed as a function of the distance between the scan plane and marking plane. The side lasers, which are mounted in front of the gantry, are often struck by the patient carts and wheelchairs, which can result in misalignment of the lasers and a delay for calibration.

The present application contemplates a new method and apparatus, which overcomes the above-referenced problems and others.

In accordance with one aspect of the present invention, a diagnostic imaging system is disclosed. The diagnostic imaging system comprises a stationary gantry; a subject-receiving bore defined in the stationary gantry; an imaging isocenter being defined centrally in the bore; first and second lasers mounted to the stationary gantry; a cover shroud covering the stationary gantry and the lasers, the shroud defining windows through which light from the lasers passes into the bore; and a couch for moving a region of interest of a subject into the bore.

In accordance with another aspect of the present invention, a method of diagnostic imaging is disclosed. A stationary gantry is provided. A subject-receiving bore is defined in the stationary gantry. An imaging isocenter is defined as being central in the bore. First and second lasers are mounted to the stationary gantry. The stationary gantry and the lasers are covered with a cover shroud. Windows in the shroud are defined, through which light from the lasers passes into the bore. A region of interest of a subject is moved into the bore.

One advantage of the present invention resides in mounting at least transverse and coronal marking lasers integrally with the scanner.

Another advantage resides in setting up the marking lasers prior to the system shipment.

Another advantage resides in reducing the mounting vulnerability of the marking lasers and thus reducing a need for re-calibration.

Another advantage resides in maintaining the marking accuracy.

Another advantage resides in reduced installation time, since the side lasers are delivered installed and calibrated in the scanner.

Yet another advantage resides in improved shielding of the lasers.

Still further advantages and benefits of the present invention will become apparent to those of ordinary skill in the art upon reading and understanding the following detailed description of the preferred embodiments.

The invention may take form in various components and arrangements of components, and in various steps and arrangements of steps. The drawings are only for purposes of illustrating the preferred embodiments and are not to be construed as limiting the invention.

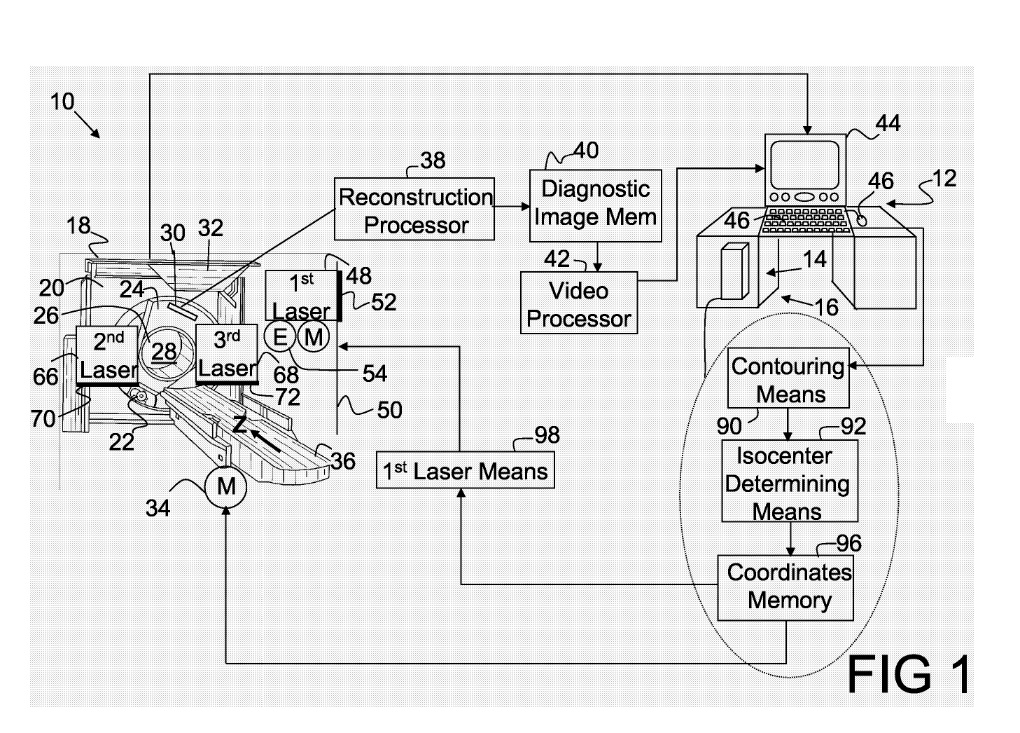

FIG. 1 is a diagrammatic illustration of an imaging system;

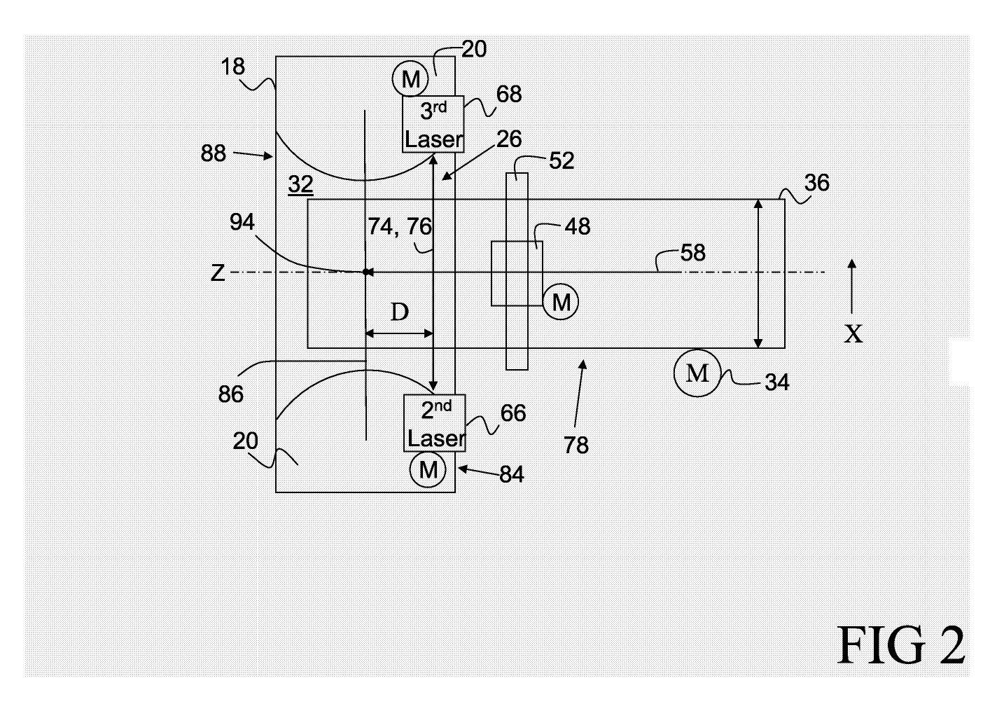

FIG. 2 is a diagrammatic illustration of a top view of the scanning area; and

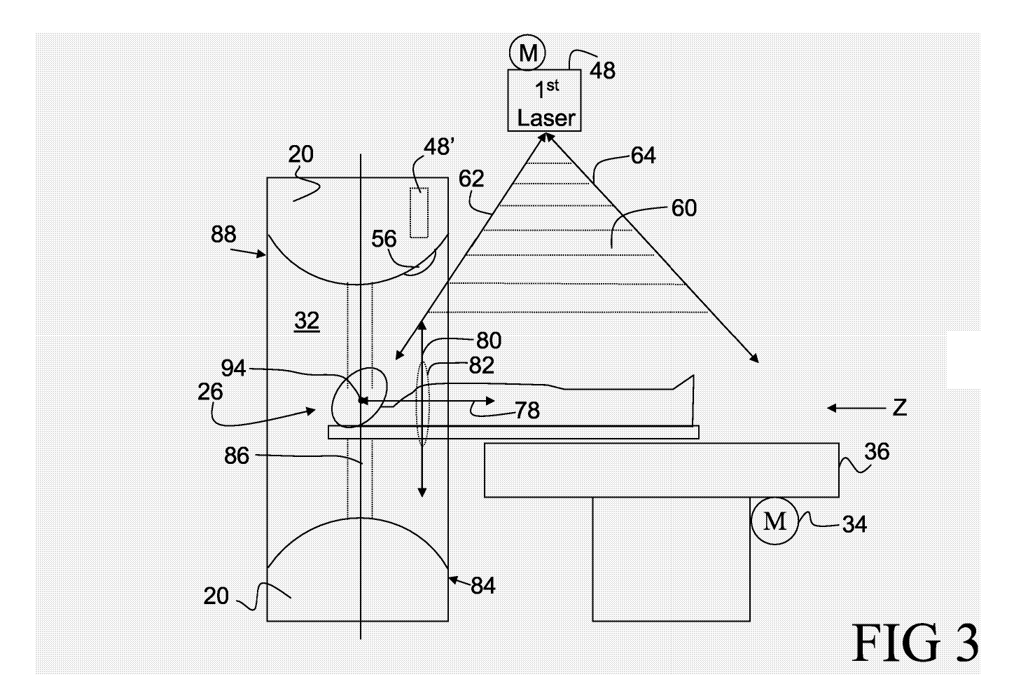

FIG. 3 is a diagrammatic illustration of a side view of the scanning area.

With reference to FIG. 1, an operation of an imaging system 10 is controlled from an operator workstation 12, which includes a hardware means 14 and a software means 16 for carrying out the necessary image processing functions and operations. Typically, the imaging system 10 includes a diagnostic imager such as CT scanner 18 including a non-rotating gantry 20. An x-ray tube 22 is mounted to a rotating gantry 24. A bore 26 defines an examination region 28 of the CT scanner 18. An array of radiation detectors 30 is disposed on the rotating gantry 24 to receive radiation from the x-ray tube 22 after the x-rays transverse the examination region 26. Alternatively, the array of detectors 30 may be positioned on the non-rotating gantry 20. The stationary and rotating gantries 20, 24 and the bore 26 are covered with a cosmetic shroud 32 that improves appearance and protects the subject and technician from moving parts, electrical components, hot parts, and the like.

Typically, the imaging technician performs a scan using the workstation 12. A couch moving means 34, such as a motor and a drive, moves a couch 36 with a subject to position the couch in the examination region 28, where an image of a region of interest of the subject is taken. The couch 36 includes drive mechanisms (not shown) which are used to move the couch 36 to a higher and lower positions with respect to the floor. Electronic data is reconstructed by a reconstruction processor 38 into 3D electronic image representations which are stored in a diagnostic image memory 40. The reconstruction processor 38 may be incorporated into the workstation 12, the scanner 18, or may be a shared resource among a plurality of scanners and workstations. The diagnostic image memory 40 preferably stores a three-dimensional image representation of an examined region of the subject. A video processor 42 converts selected portions of the three-dimensional image representation into appropriate format for display on one or more video monitors 44. The operator provides input to the workstation 12 by using an operator input device 46, such as a mouse, touch screen, touch pad, keyboard, or other device.

With continuing reference to FIG. 1 and further reference to FIGS. 2 and 3, a first or saggital laser 48 is mounted to a wall or ceiling 50 via a first mounting means 52. The mounting means 52 moves the saggital laser transversely to position its vertical beam directly over a selected plane of the subject. An encoder 54 measures the transverse location of the saggital laser 48. Of course, it is also contemplated that the saggital laser 48 can be overhead mounted on an extension arm, and the like. In one embodiment, where the saggital laser 48′ is mounted to the stationary gantry 20 of the scanner, transversely elongated window 56 is defined in the shroud 32 for a laser line to reach the subject. The saggital laser 48 generates a line 58 along the axial direction Z in a vertical plane 60 which extends vertically through or parallel to the Z-axis and is circumfused by laser rays 62, 64.

Second and third or side lasers 66, 68 are mounted firmly to the stationary gantry 20 via associated second and third mounting means 70, 72 which move the lasers 66, 68 vertically in a common plane. The side lasers 66, 68 generate laser lines 74, 76 in a horizontal transverse plane 78 and a vertical transverse plane 80, both perpendicular to and intersecting the saggital vertical plane 60 to define crosshairs on the sides of the subject. The vertical plane 78 intersects the vertical, longitudinal saggital plane 60 on an upper surface of the subject. The shroud 32 has a vertical window 82 for each side laser 66, 68. Preferably, the side lasers 66, 68 are disposed in a close proximity to a front 84 of the gantry 20 such that the distance D between a scanning plane 86 and the horizontal plane 78 generated by the lines of lasers 66, 68 is approximately 50-200 mm. Because the side lasers 66, 68 are positioned at a minimal distance to the scanning plane 86, the marking accuracy is maintained with fewer requirements for the positioning of the patient support in terms of repeatability and accuracy.

In one embodiment, the side lasers 66, 68 are mounted close to a rear 88 of the bore 26 or a second set of lasers is mounted close to the rear.

With continuing reference to FIG. 1, the contouring means 90 segments the 3D image to delineate a specific anatomical target volume within the region of interest such as a tumor. The target boundary is adjusted by the user by a use of the input means 46. An isocenter determining means 92 determines an isocenter 94 of the contoured volume, e.g. a center of mass of the tumor to be treated, which is stored in a coordinates memory 96.

After the scanning operation is completed, the isocenter coordinates x, y, z, which have been determined by the isocenter determining means 92, are used by the operator or a software routine at the workstation 12 to move the couch 36 and/or the lasers 48, 66, 68 accordingly up and down, and/or in and out. More specifically, the moving means 34 positions the couch 36 and the side lasers 66, 68 are moved up or down as necessary such that the side lasers 66, 68 project their crosshairs on the side of the subject directly in line with the center of mass 94 of the tumor. The laser mounting means 52 moves the saggital laser 48 left or right such that the saggital laser's line 58 intersects the center of mass 94. The laser projections provide three crossing points: one on each side of the subject and a third one on the top of the subject where the crosshairs of the side lasers 66, 68 intersect the longitudinal line 58 of the saggital laser 48. While the laser lines are projected onto the subject in accordance with the determined isocenter 94 of the tumor, the small dots are placed on each of the crossing points to mark the isocenter 94 and provide for reproducible positioning of the subject with respect to the isocenter of the x-ray source 22 during the radiotherapy sessions.

Rather than positioning the second and third lasers 66, 68 at 3 and 9 o'clock, the second and third lasers 66, 68 can be positioned at other angles.

The invention has been described with reference to the preferred embodiments. Modifications and alterations will occur to others upon a reading and understanding of the preceding detailed description. It is intended that the invention be construed as including all such modifications and alterations insofar as they come within the scope of the appended claims or the equivalents thereof.

1. A diagnostic imaging system comprising:
a stationary gantry;
a subject-receiving bore defined in the stationary gantry;
an imaging isocenter being defined centrally in the bore;
first and second lasers mounted to the stationary gantry;
a cover shroud covering the stationary gantry and the lasers, the shroud defining windows through which light from the lasers passes into the bore; and
a couch for moving a region of interest of a subject into the bore.

2. The system as set forth in claim 1, further including:
a rotating gantry for rotating an x-ray source about a longitudinal axis to define a scanning plane.

3. The system as set forth in claim 2, wherein the first and second lasers are mounted a distance between 50 mm and 200 mm from the scanning plane.

4. The system as set forth in claim 2, wherein the first and second lasers are mounted a distanced, which is equal to one half of a distance from the scanning plane to a bore front entrance.

5. The system as set forth in claim 1, wherein the first and second lasers are integrally mounted adjacent one of a front entrance Band a rear side of the bore.

6. The system as set forth in claim 1, further including:
a means for segmenting the image to outline at least an organ; and
a means for determining an isocenter of the segmented organ; wherein the first and second lasers laser mark the subject based on the determined isocenter.

7. The system as set forth in claim 6, wherein the first and second lasers each projects a side line on sides of the subject in a horizontal plane which is perpendicular to a vertical plane.

8. The system as set forth in claim 7, further including:
a moving means for adjusting the subject couch into which moving means coordinates of the isocenter are loaded from a memory Rand which moving means adjusts the couch such that the side lines are projected in line with the segmented organ.

9. The system as set forth in claim 8, further including:
a saggital laser for laser marking the subject based on the determined isocenter, which saggital laser is mounted externally to the scanner and projects a longitudinal line on a top of the subject in the vertical plane which is parallel to an axial direction.

10. The system as set forth in claim 9, further including:
a laser mounting means for adjusting at least one of the saggital, first and second lasers concurrently with the moving means adjusting the couch such that the lines projected by the saggital, first and second lasers intersect the determined isocenter.

11. A method of diagnostic imaging comprising:
providing a stationary gantry;
defining a subject-receiving bore in the stationary gantry,
defining an imaging isocenter as being central in the bore;
mounting first and second lasers to the stationary gantry;
covering the stationary gantry and the lasers with a cover shrouds,
defining windows in the shroud, through which light from the lasers passes into the bore; and
moving a region of interest of a subject into the bore.

12. The method as set forth in claim 11, further including:
rotating an x-ray source Ron a rotating gantry about a longitudinal axis; and
defining a scanning plane.

13. The method as set forth in claim 12, wherein the step of mounting includes:
mounting the first and second lasers a distance between 50 mm and 200 mm from the scanning plane.

14. The method as set forth in claim 11, wherein the step of mounting includes:
mounting the first and second lasers integrally adjacent at least one of a front entrance and a rear side of the stationary gantry of the scanner.

15. The method as set forth in claim 11, further including:
segmenting the image to outline at least an organ;
determining an isocenter of the segmented organ; and
laser marking the subject based on the determined isocenter.

16. The method as set forth in claim 15, further including:
projecting lines on sides of the subject in a horizontal plane which is perpendicular to an axial direction (Z)with the first and second lasers.

17. The method as set forth in claim 17, further including:
projecting a longitudinal line on a top of the subject in a vertical plane which is parallel to the axial direction with a saggital laser mounted above the subject.

18. The method as set forth in claim 18, further including:
laser marking the subject with the saggital laser.

19. The method as set forth in claim 18, further including:
concurrently adjusting the couch and one or more of the lasers such that the lines projected by the saggital, first and second lasers intersect the determined isocenter.

20. A diagnostic imaging system to perform the steps of the method of claim 11, and further including:
a radiotherapy planning workstation.
